(19) United States
(12) Patent Application Publication
(10) Pub. No.: US 2005/0021133 A1
(43) Pub. Date: Jan. 27, 2005

(54) Tubular stents sandwiched inside of a composite membrane and methods of making and using thereof

(76) Inventor: Dong Li, Creve Coeur, MO (US)

Correspondence Address: Dong Li, 681 Questover Ln, Creve Coeur, MO, 63141, US

(21) Appl. No.: 10/623,935

(22) Filed: Jul. 22, 2003

(51) Int. Cl. A61F 2/06

(52) U.S. Cl. 623/1.44

(57) ABSTRACT

A novel modification method for commercially available tubular stent is invented. The modified stents are sandwiched between a composite membrane. The polymeric sandwich (composite membrane) is durable, which undergoing several crimping and expansion circles without broken nor pinhole. The modified stents are very useful to replace the stent-graft in the procedure of aneurysm rupture prevention. The outside layer polymer has mechanical advantages such as high degree of elasticity, excellent durability and having been approved for some clinic application. The inside polymeric layer is covalently bonded to the outside polymeric layer and at the same time is cross-linking with itself. Not only this layer of polymer is biocompatible, but also it requires limited smooth muscle cells' proliferation. In some cases, the inside polymeric layer can be used as a platform of control drug release device. The modification method could be used to produce both large stent applicable in large vessels(greater or equal to 3 mm diameter) and small stent applicable in small vessels (less than 3.0 mm diameter and can be crimped on a 1.5 mm angioplasty balloon catheter), and to produce customer length tubular stent.

### REFERENCE

1. Shimada, Atsushi; Nagura, Hiroaki. “Medical implants coated with fluorine-containing diamond-like carbon film” Feb. 6, 2001. Japan Patent 2001029447

2. Fujii, Toshikatsu; Abe, Masanori; Miyawaki, Fujio. “Stent with ferrite membrane” Feb. 13, 2001, Japan Patent 2001037866

3. Multanen, Markku; Talja, Martti; Hallanvuo, Saija; Siitonen, Anjia; Valimaa, Tero; Tammela, Teuvo L. J.; Seppala, Jukka; Tormala, Pertti. “Bacterial adherence to silver nitrate coated poly(L-lactic acid) urological stents in vitro” (2000) Urological Research, 28(5), 327-331

4. Zhong, Sheng-ping. “Hybrid Coating for medical devices” Jan. 30, 2001. U.S. Pat. No. 6,179,817

5. Ruebben, Alexander. “Magnetic-resonance-compatible balloon-expandable endoprosthesis (MR Stent) made from a high-gold-content alloy” Mar. 29, 2001 DE 29917261

6. Leclerc, Guy; Fareh, Jeannette; Leblanc, Philippe; Levesque, Lue; Martel, Remi; Kudrevich, Svetlana; Lawrence, Marcus F.; Bourguigon, Bernard; Lessard, Jean; Blais, Sonia; Chapuzet, Jean-Marc; Meunier, Michel; Napporn, Teko; Poulin, Suzie; Sacher, Edward; Savadogo, Oumarou. “Radioactively coated device and method of making same for preventing restenosis” Mar. 1, 2001, WO 2001014617

7. Marton, Denes. “Self-supportting laminated films, structural materials and medical devices manufactured therefrom and methods of making same” Nov. 22, 2001, WO 2001087371

8. Jadhav, Balkrishna S. “Bioresorbable stent” Apr. 9, 2002, U.S. Pat. 6,368,346

9. Lau, Lilip; Maroney, Charles Thomas; Hartigan, William M.: Rhee, Woonza; Mccullough, Kimberly A. “Self-expandable helical intravascular stent and stent-graft” Dec. 26, 2000, U.S. Pat. No. 6,165,210

10. Acciai, Michael A.; Hall, Richard R.; Legg, John T. “Method and tooling for forming a stent” Feb. 27, 2001, U.S. Pat. No. 6,193,829

11. Schering A.-G. “Stents composed of shape memory polyurethanes and incorporating active substances” Mar. 28, 2002, DE 20119322

12. Koole, Levinas H.; Binderman, Itzhak. “Method for immobilizing poly(HEMA) on stents” Mar. 28, 2002, WO 2002024249

13. Trozera, Thomas. “Method of manufacturing a stent” May 11, 1999, U.S. Pat. No. 5,902,475

14. Archodoulakis, Georgios. “Highly flexible implant for intro and/or endovaskulare applications (Stent) and manufacturing processes [machine translation]” Apr. 4, 2002, DE 10045325

### BACKGROUND OF THE INVENTION

Stenting is a catheter-based procedure in which a stent (a small, expandable metal wire mesh tube) is inserted into a diseased artery to hold it open. Currently, stenting is performed most often in conjunction with other catheter-based procedures, such as balloon angioplasty or atherectomy. The angioplasty procedure reduces the narrowing of the coronary artery, then, the stent is immediately inserted. Therefore, the stent typically allows for an excellent final result to be obtained with little to no narrowing remaining within the coronary arteries. Stents may also be used to restore normal blood flow in arteries that have been torn or otherwise damaged. In addition to treating the coronary arteries, stents may be inserted in many other arteries in the body. For example, they are commonly used to support and hold open arteries in the kidney (renal arteries) or the iliac arteries that supply blood to the legs, and in the carotid arteries, which supply oxygen-rich blood to the brain.

In order to use the stent, a corresponded stent-delivery system should be considered. A stent with desired properties, such as diameter, length, texture, chemical properties will be crimped onto a PTCA (percutaneous transluminal coronary angioplasty) balloon with a guide wire. The balloon catheter loaded with the stent is introduced through the artery and inflated at certain pressure for short period of time to expand the stent against the arterial wall. Therefore, it requires both commercial available stents and any modified stents to have crimping ability and dimension stability after expansion. For example, the diameter of a stent could be reduced 60% from un-crimped to crimped, and the diameter of the same stent could be increase 400% from crimped to expanded. Even stents come in a variety of different texture, plasticity, elasticity, strength, diameter size, chemistries and other properties; but the crimping ability and dimension stability are always properties required for stenting application.

Stents are permanent devices after stenting operation, stents in essence become part of the cardiovascular system. Stents carry a risk of two long-term complications. First, there is the possibility of recurrence of narrowing at the site of stent placement (restenosis). Second, there is a risk of a blood clots (thrombus) forming in the stent. Although multiple complex biology pathways are co-operatively triggered to results in excessive neoarterial tissue growth (intimal hyperplasia), the subsequent cellular events must be carefully considered. That is, minimal occurrence of thrombus, associated with mechanic vascular injury during stent dilation or initiated from the stents surface after stenting, reducing proliferation of smooth muscle cell and rapid re-endothelialization are used to critically evaluate the stenting procedure and stent modification. In order to meet these requirements, several approaches have been pursued, such as the completely flexible diamond-like carbon coating^{[1]}, stent with ferrite membrane^{[2]}, silver nitrate coated poly(L-lactic acid) ^{[3]}and hybrid coating^{[4]}. Other advantage for this surface coating method with different chemotherapeutic bio-compatible material or drugs is to potentially minimize resteosis. Other examples are stents made from a high-gold-content alloy^{[5]} and charged radioactively coated material ^{[6]}.

Clinic studies show the extent of neointima formation correlates with vessel wall injury, which occurred during the stent introducing procedure and/or stent expansion. Therefore, some inventions focused on surface finish with rounded edges^{[8]}, electrochemical polishing^{[9]} and silicone-covered stents^{[10]} and stent grafts^{[11]}.

Another important application for stent is combined with graft, called as stent-graft procedure. Physicians use stent-graft procedure as a new endo-vascular technology to treat aneurysms with minimally invasive surgery, such as cerebral aneurysm or abdominal aortic aneurysm.

An aneurysm is a bulge in the blood vessel. When a normal blood vessel is stressed, it undergoes immediate elastic deformation, which is greater when the stress is higher. In the first short period of time, it makes additional, relatively rapid, plastic adjustments at points of stress. These initial plastic adjustments give way to a slow, nearly steady rate of strain, like the creep in the behavior of polycrystalline metals. The steady creep continues over an extended period of time, until sufficient strain has developed so that a necking-down and area-reduction occurs. After the creep behavior was occurred, two continue processes could cause the rupture. If a constant load was applied, the rate of strain accelerated until rupture occurs, or if the load was adjusted to maintain a constant stress, the creep rate would continue until rupture. Therefore, aneurysm(s) is relatively common and affect the larger arteries throughout the body, even in the brain. Even it is not clear why a person develops an aneurysm, which might related to an absence of a muscular layer makes up part of the blood vessels, but it is certain that aneurysm can and do grow. When the aneurysm grows bigger, it can break open and bleed into the surrounding area. In cerebral aneurysm, the aneurysm breaks and bleed is called a hemorrhage or rupture. There are several post-bleed problems, such as re-bleeding, hydrocephalus (water on the brain) and vasospasm (narrowing of the blood vessels because of the irritation of the blood on the blood vessels), can happen days to weeks after the initial bleed. Therefore, some treatments prevented the rupture should be pursued.

If the aneurysm reaches a certain size, it can start putting pressure on the surrounding area and cause progressive problems. It is like a balloon, when it is pressured, it is always expanded and stretched from the weakest point. The aneurysm is the weakest point along the blood vessel. Without enforcement at the weakest point, the aneurysm will grow and become even weaker than before. Therefore, an idea treatment is to reduce the local pressure on the aneurysm without reduce the blood flow. Physicians perform endovascular surgery using stent-graft by navigating a small tube or catheter into the aneurysm, then placing one stent just above the aneurysm and a second stent just below the aneurysm. The two stent are connected by a patch of synthetic material (a graft) to provide a channel for blood to flow without entering the aneurysm. In the previous stent-graft procedure, the two stent are used only to fix the graft to the certain position through expanding the stents against the blood vessel wall, and the graft is used as a channel for blood to flow without entering the aneurysm. In order to do this, the all four sizes (diameter) of the blood vessel from patient's aneurysm, catheter used to deliver the stent-graft, stents and graft should be accommodated. Since the stent can be crimped but the graft is not, in most cases, the doctor has difficulty placing the graft through your arteries, the doctor may need to perform some additional surgery in order to get the stent-graft into place. Therefore, one piece “stent-graft”, which is crimp able and expandable without broken the graft, even a pinhole, is desired for investigation. Furthermore, the stent sandwiched inside a composite membrane could work as the enforced material to prevent the deformation of the graft.

The sandwich type modified stent not only just undergoing the crimping and expansion, after expanding, but also the outside layer polymer is still unbroken, even without pinhole. These requirements are very useful to treat the aneurysm. The advantages of stent sandwiched inside of composite membrane to treat aneurysm are that the stent is only one piece, which will fix the outside layer to the certain position through expanding the stent against the blood vessel wall and enforce the outside layer strength to provides a channel for blood to flow without entering the aneurysm without pressurizing the aneurysm nor deforming, such as collapsing which will cause the irritation of the blood on the blood vessels. The composite membrane sandwiched with a stent will required its mechanic properties less than that of the stent-graft case, therefore, it could be such thin as long as the outside layer is a perfect film without pinhole.

Accordingly, the aim of the present invention is to create a modification method of commercially available tubular stent. The modified stents are sandwiched between a composite membrane. The modification method will take into account of material design, including polymeric material with special chemical and mechanic properties, and surface design, including immobilization of drugs and coating of polymers and those hydrogels impregnating with drugs and peptides for sustained drug release.

The polymeric sandwich (composite membrane) should be durable undergoing the crimping and expansion circle. The outside layer polymer has mechanical advantages such as high degree of elasticity, excellent durability and having been approved for some clinic application. Inside polymeric layer is covalently bonded to the outside polymeric layer and at the same time is cross-linking with itself. Not only this layer of polymer is biocompatible, but also it requires limited smooth muscle cells' proliferation. In some cases, the inside polymeric layer can be used as a platform of control drug release device. Furthermore, the method could be used to produce both large stent applicable in large vessels (greater or equal to 3 mm diameter) and small stent applicable in small vessels (less than 3.0 mm diameter and can be crimped on a 1.0 mm angioplasty balloon catheter). It is therefore an objective of the present invention to provide a method to produce customer length tubular stent.

### SUMMARY OF THE INVENTION

The present invention provides a modification method of commercially available tubular stent. The modified stents are sandwiched between a composite membrane. Another aspect of the present invention provides modification method will take into account of material design, including polymeric material with special chemical and mechanic properties, and surface design, including immobilization of drugs and coating of polymers and those hydrogels impregnating with drugs and peptides for sustained drug release. The polymeric sandwich (composite membrane) should be durable undergoing the crimping and expansion circle.

In another embodiment, the outside layer polymer has mechanical advantages such as high degree of elasticity, excellent durability and having been approved for some clinic application. Inside polymeric layer is covalently bonded to the outside polymeric layer and at the same time is cross-linking with itself. Not only this layer of polymer is biocompatible, but also it requires limited smooth muscle cells' proliferation. In some cases, the inside polymeric layer can be used as a platform of control drug release device. Furthermore, the method could be used to produce both large stent applicable in large vessels(greater or equal to 3 mm diameter) and small stent applicable in small vessels (less than 3.0 mm diameter and can be crimped on a 1.5 mm angioplasty balloon catheter).

In another embodiment, the present invention provides a method to produce customer length tubular stent.

### DETAIL DESCRIPTION OF THE INVENTION

The present invention provides a modification method of commercially available tubular stent. The modified stents are sandwiched inside a polymeric composite membrane. The outside layer of polymer, which will face the artery wall, is form parallel to stent surface and completely cover the stent and two ends of the tube. The inside layer of polymer, which will form the luminal surface, is formed perpendicular to the stent surface and cover the stent. The outside layer and inside layer are different polymers characterized with total different chemical and mechanic properties. For example, the outside layer polymer requires high degree of elasticity and durability, except bio-compatibility. The high degree of elasticity of the outside layer polymer is useful in all extents, which including to smooth the stent surface and edges, which will dramatically prevent the lesion in the introduction and expansion of stent and to change topography of the edge area adjacent to where the stent wire is presented. Durability favors low profiles because an extremely thin layer of polymer can be utilized, therefore not only the polymeric layer thickness in the contraction is small which will let the stent catheter introduction easy, but also the polymeric layer extras into a thin film fastened to the inner surface of the stent platform by adhesion. For example, the inside layer polymer requires high degree of promoting selectively one kind of cells attachment to a polymer scaffold in the presence of other kinds of cells in order to reduce the possibility of recurrence of narrowing at the site of stent placement (restenosis), and reduce the risk of a blood clots (thrombus) forming in the stent.

The silicones are the series polysiloxane commercially available, which are widely used in the manufacture of cosmetics, food-processing materials and medicinal preparation. They are valued for their high degree of elasticity and durability in this invention.

Differences in materials recommendations depend primarily on manufacturing practice and control. The fabrication technique associated with outside layer stent is coating. Selection of a particular silicone is dictated both by its fabrication and performance characteristics.

A variety of groups, including phenyl, vinyl, and hydrogen can substitute for the methyl group in a polydimethylsiloxanes. A variety of groups, including methylacryloxypropyl, and acryloxy can replace the termination unit, the trimethylsiloxy group (Me3SiO) in polysiloxane copolymer. These substituted groups could dictate the conditions by which polysiloxane curing or cross-linking can be accomplished.

In room temperature vulcanizing system, one of the following three methods are used:
1. Silanols are condensed,
2. A metal salt catalyzes the reaction between silicon hydrides and silanols;
3. A platinum complex catalyzes the vinyl addition of silicone hydrides;

Therefore, it is idea to cross-link the silicone of the outside layer and covalently bond the inside layer of the polymer. For example, a platinum complex catalyzes the vinyl addition of silicones hydrides and the vinyl addition of acryloxy or methylacryloxy monomer or pre-polymer.

It is a strong desire to discover small ligands that can promote selectively one kind of cells attachment to a polymer scaffold in the presence of other kinds of cells. Studies of Elbert et al. demonstrated a number of polyethylene glycol-based (PEG) materials that resist protein adsorption and which remained non-adhesive to cells even in the presence of serum proteins or blood. It is an objective of the present invention to use polyethylene glycol-based acryl aid (PEGA) or polyethylene glycol-based diacryl aid (PEGDA) formed the luminal surface of the stent, which is biocompatible and resist protein adsorption and which remained non-adhesive to cells even in the presence of serum proteins or blood. The polyethylene glycol-based diacryl aid (PEGDA) itself can cross-link under certain conditions. The state-of-art of this present invention is to control the sequence of cross-linking. The silicone is cross-linked first during the period of curing, then the silicone is covalently bonded with PEGA or PEGDA, finally, the PEGDA is cross-linked itself to form the inside layer of composite membrane.

### EXAMPLE 1

Palmaz Bolloon expandable stent P308A (un-crimped outside diameter of 3.40 mm and inside diameter of 2.31 mm, original length of 30.0 mm) inserted into a solid bar (with outside diameter of 2.40 mm, length of 100.0 mm). The outside diameter of the bar is little bigger than the inside diameter of the stent in order to hold the stent from free fall even if the stent and bar in a vertical position. Lock the bar with the stent into a motor, which can be controlled for the rotation speed. Check the bar for prefect vertical position, which will let the evenness of the coating film.

The mixture is pour on the bar top, the polysiloxane solution will fall down slowly due to the gravity force while the centrifuge force from the bar rotation will coat the film around the stent. The buoyancy force of the bubble will let the bubbles left the solution into the surface. In general, when these four kinds of forces (viscosity, gravity, centrifuge and buoyancy) reach equilibrium while the polysiloxane is curing, a film is form.

After the polysiloxane is cured, unlock the bar from the motor and cut the film along the two ends of the stent but left a little space from the tips of the stent in order to let the edges of the stent ends are coated with polymer. Then take out the tubular stent with outside layer of polymer. Coat the precursor of polymer solution B inside the stent only. After the precursor of polymer B is polymerized with UV light, a stent sandwiched inside of a composite membrane is formed. The outside layer of stent is coated with polysiloxane derivative, and inside layer of stent is coated with PEGA or PEGDA. The outside layer is covalently bonded with inside layer. The stent is ready to crimp on the balloon catheter easily. The stent is crimped on a balloon catheter with outside diameter of 3.0 mm. Later on, the stent is expanded into 5.6 mm without broken the film.

### EXAMPLE 2

Multi-link Penta coronary stent system (un-crimped outside diameter of 2.75 mm and inside diameter of 2.31 mm, original length of 30.0 mm) inserted into a solid bar (with outside diameter of 2.40 mm, length of 100.0 mm). The outside diameter of the bar is little bigger than the inside diameter of the stent in order to hold the stent from free fall even if the stent and bar in a vertical position. Lock the bar with the stent into a motor, which can be controlled for the rotation speed. Check the bar for prefect vertical position, which will let the evenness of the coating film.

The mixture is pour on the bar top, the polysiloxane solution will fall down slowly due to the gravity force while the centrifuge force from the bar rotation will coat the film around the stent. The buoyancy force of the bubble will let the bubbles left the solution into the surface. In general, when these four kinds of forces (viscosity, gravity, centrifuge and buoyancy) reach equilibrium while the polysiloxane is curing, a film is form.

After the polysiloxane is cured, unlock the bar from the motor and cut the film along the two ends of the stent but left a little space from the tips of the stent in order to let the edges of the stent ends are coated with polymer. Then take out the tubular stent with outside layer of polymer. Coat the precursor of polymer solution B inside the stent only. After the precursor of polymer B is polymerized with UV light, a stent sandwiched inside of a composite membrane is formed. The outside layer of stent is coated with polysiloxane derivative, and inside layer of stent is coated with PEGA or PEGDA. The outside layer is covalently bonded with inside layer. The stent is ready to crimp on the balloon catheter easily. The stent is crimped on a balloon catheter with outside diameter of 1.5 mm. Later on, the stent is expanded into 4.0 mm without broken the film. While the foregoing is directed to the preferred embodiments of the present invention, other and further embodiments of the invention may be devised without departing from the basic scope, which is determined by the following claims:

1. A modification method of sandwiching a commercially available tubular stent with a polymeric composite membrane is invented.

2. The polymeric composite membrane of claim 1, wherein is durable, which undergoing several crimping and expansion circles without broken nor pinhole.

3. The polymeric composite membrane of claim 1, wherein is two layers. The outside layer polymer has mechanical advantages such as high degree of elasticity, excellent durability and having been approved for some clinic application.

4. The polymers of the outside layer of the polymeric composite membrane of claim 1, wherein are silicon, poly(ethylene) glycol, polyurethane or polyurea or their derivatives.

5. The inside polymeric layer of the polymeric composite membrane of claim 1, wherein is, covalently and vertically, bonded to the outside polymeric layer and at the some time is cross-linking with itself.

6. The polymers of the inside layer of the polymeric composite membrane of claim 1, wherein are silicon, poly(ethylene) glycol, polyurethane or polyurea or their derivatives.

7. Not only this layer of polymer is biocompatible, but also it requires limited smooth muscle cells' proliferation. In some cases, the inside polymeric layer can be used as a platform of control drug release device.

8. Another aspect of the present invention provides that the modification method could be used to produce both large stent applicable in large vessels(greater or equal to 3 mm diameter) and small stent applicable in small vessels (less than 3.0 mm diameter and can be crimped on a 1.5 mm angioplasty balloon catheter), and to produce customer length tubular stent.
